(19) United States
(12) Patent Application Publication
(10) Pub. No.: US 2021/0219896 A1
(43) Pub. Date: Jul. 22, 2021

(54) ELECTRODE FOR RECORDING A PHYSIOLOGICAL ELECTRICAL SIGNAL, ASSOCIATED SENSOR AND DEVICE

(71) Applicant: CONSCIOUS LABS SAS, LIMOGES (FR)

(72) Inventors: Julien DAUGUET, Paris (FR); Alexandre BOULLE, Paris (FR); David MARDIVIRIN, Saint-Priest-Sous-Aixe (FR)

(21) Appl. No.: 15/734,350

(22) PCT Filed: Jun. 5, 2019
(86) PCT No.: PCT/FR2019/051347

§ 371 Date: Dec. 2, 2020

(30) Foreign Application Priority Data
Jun. 5, 2018 (FR) 1854851

(51) Int. Cl. A61B 5/291

(52) U.S. Cl. CPC A61B 5/291; A61B 2562/164; A61B 2562/0209

(57) ABSTRACT

The electrode for recording a physiological electrical signal of a living being made from electrically conductive material and including a body having a front surface and a rear surface for electrical contact, the electrode further including a rigid mounting element attached to the body, at an edge of the rear electrical contact surface and designed so as to produce a removable mounting of the electrode on a recording sensor.

### TECHNICAL FIELD OF THE INVENTION

The invention relates to an electrode for recording a physiological electrical signal of a living being, particularly an electrical signal corresponding to an electroencephalograph (EEG) signal. The invention also relates to a recording sensor comprising such an electrode and to a recording device comprising such a sensor.

### PRIOR ART

At the present time, there are polymer electrodes which are soft and comfortable, which are fixed solidly to a printed circuit of a recording sensor for recording a physiological electrical signal in the context of electroencephalograms or electrocardiograms. Such electrodes are described in the publication by Yun-Hsuan CHEN et al., “Soft, Comfortable Polymer Dry Electrodes for High Quality ECG and EEG Recording”, Sensors 2014, 14, pp. 23758-23780. However, such sensors require the entirety of the sensor to be replaced/cleaned between each patient or else when the polymer electrode needs to be changed for reasons of wear, hygiene, location, hair type, etc. This leads to high costs.

### SUMMARY OF THE INVENTION

It is an object of the invention to provide a recording electrode for a recording sensor which does not exhibit the abovementioned disadvantages.

To this end, the invention provides an electrode for recording a physiological electrical signal of a living being made from an electrically conducting material and comprising a body comprising a front surface and a rear surface for electrical contact, the electrode further comprising a rigid mounting means fixed to the body, at an edge of the electrical-contact rear surface and arranged in such a way as to achieve removable mounting of the electrode on a recording sensor.

Advantageously, but optionally, the electrode according to the invention exhibits at least one of the following technical features:
the material is a soft electrically conducting material, notably of polymer type;
the rear surface of the body is convex;
the rigid mounting means comprises a cylindrical ring fixed attached to the body;
the ring comprises a screw thread on a radially external surface;
the ring projects from the rear surface of the body;
the rigid mounting means comprises a stiffening washer;
the electrode further comprises a tool socket allowing the removable mounting; and
the electrode further comprises pins extending as a projection from the front surface.

The invention provides a sensor for recording a physiological electrical signal of a living being comprising a casing comprising a housing, a printed circuit positioned in the housing, and an electrode, exhibiting at least one of the abovementioned technical features, mounted removably on the casing, the rear surface pressing with electrical contact against a part of the printed circuit.

Advantageously, but optionally, the sensor according to the invention exhibits at least one of the following technical features:
the casing comprises an assembling means complementing the rigid mounting means of the electrode so that the electrode can be mounted removably on the sensor; and
the casing comprises a means forming shielding designed to achieve electromagnetic isolation.

The invention also provides a device for measuring a physiological electrical signal intended to be worn by a living being comprising at least one sensor exhibiting at least one of the abovementioned technical features.

### BRIEF DESCRIPTION OF THE FIGURES

Further features and advantages of the invention will become apparent from reading the following description of one embodiment of the invention. In the attached drawings:

FIG. 1 is a view in section of a first embodiment of a recording sensor according to the invention;

FIG. 2 is a view in section of a second embodiment of a recording sensor according to the invention;

FIG. 3 is a view in section of the sensor of FIG. 2 associated with an instrument for mounting/demounting the recording electrode; and

FIG. 4 is a three-dimensional view of the sensor of FIG. 2 associated with the mounting instrument of FIG. 3.

For greater clarity, elements which are identical or similar are identified by identical reference signs across all the figures.

### DETAILED DESCRIPTION OF AN EMBODIMENT

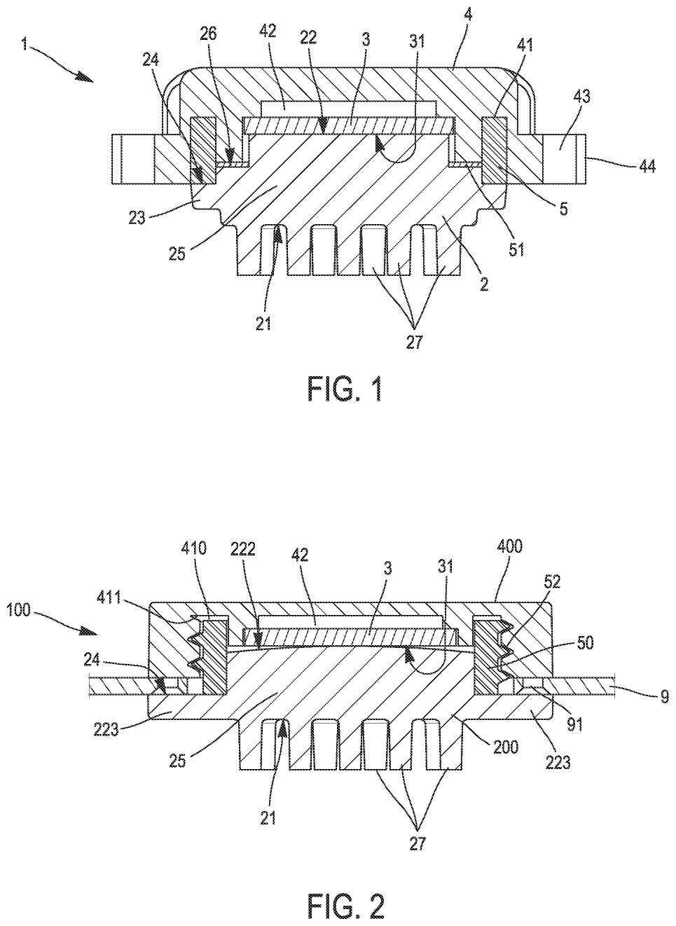

A first embodiment of a sensor 1 for recording a physiological electrical signal of a living being according to the invention, associated with a first embodiment of an electrode 2 for recording a physiological electrical signal of a living being according to the invention, will be described with reference to FIG. 1.

The recording sensor 1 for recording a physiological electrical signal of a living being according to the invention comprises a casing 4 internally delimiting a housing 42 intended to accept a printed circuit 3. The casing 4 further comprises an assembling means 41, in this instance in the form of an annular groove surrounding the housing 42. Furthermore, the casing 4 comprises a protrusion 44 extending laterally way from the center from an external periphery and in which there is formed a through-hole 43 which allows the insertion of means for fixing the recording sensor 1 for recording a physiological electrical signal of a living being according to the invention onto a device 9 for measuring a physiological electrical signal intended to be worn by a living being. Such a device is illustrated symbolically in FIGS. 1 to 4. The protrusion 44 may take the form of an annular flange surrounding the casing 4 in which there is formed one or more through-holes 43 which are uniformly distributed on the flange. In another embodiment variant, the protrusion 44 comprises one or more lugs uniformly distributed over the external periphery of the casing 4, each of the lugs comprising a through-hole 43. In one embodiment variant, the casing 4 comprises a means forming shielding. This shielding-forming means makes it possible to achieve electrical isolation from the electromagnetic noise external to an environment in which the recording sensor is being used, such external electromagnetic noise disturbing the physiological electrical signal measured by the recording sensor.

The printed circuit 3 comprises all the components necessary for evaluating the measured physiological electrical signal and processing same so as to supply information that can be used and exploited by an external analysis and recording system (not depicted) to which the recording sensor 1 for recording a physiological electrical signal of a living being according to the invention is connected. In particular, the printed circuit comprises a contact face 31 arranged in such a way as to transmit the physiological electrical signal to the rest of the components of the printed circuit 3.

The recording sensor 1 for recording a physiological electrical signal of a living being according to the invention further comprises a recording electrode 2 for recording a physiological electrical signal of a living being according to the invention. The recording electrode 2 for recording a physiological electrical signal of a living being according to the invention is mounted removably on the recording sensor 1 for recording a physiological electrical signal of a living being according to the invention. Such removable mounting of the recording electrode 2 for recording a physiological electrical signal of a living being according to the invention on the recording sensor 1 for recording a physiological electrical signal of a living being according to the invention makes it possible to replace only the recording electrode 2 for recording a physiological electrical signal of a living being according to the invention without the need to replace the sensor in its entirety. The recording electrode 2 for recording a physiological electrical signal of a living being according to the invention comprises a main body 25 of cylindrical shape, particularly shaped as a cylinder of revolution. The body 25 comprises a rear surface 22 and a front surface 21. The rear surface 22 is an electrical-contact rear surface and, once the recording electrode 2 for recording a physiological electrical signal of a living being according to the invention is mounted on the recording sensor 1 for recording a physiological electrical signal of a living being according to the invention, presses against the contact face 31 of the printed circuit 3. This makes it possible to ensure electrical conductivity between the recording electrode 2 and the printed circuit 3 for the purposes of transmitting the measured physiological electrical signal.

In addition, the recording electrode 2 for recording a physiological electrical signal of a living being according to the invention comprises, in this instance, a series of pins 27 which extend as a projection from the front surface 21 of the body 25. These pins 27 are flexible and formed as one with the body 25. The pins 27 are intended, at a free end, to come into contact with an area of skin of the living being in order to record a physiological electrical signal that is produced beneath this area of skin. The purpose of the pins 27 is, in this instance, to optimize contact with the area of skin of the living being in the presence of keratinous fibers such as the hair or body hair. According to an embodiment variant, the front surface 21 of the body 25 is arranged in such a way as to come into contact directly with the area of skin of the living being in order to measure a physiological electrical signal that is produced beneath said area of skin.

Furthermore, the body 25 of the recording electrode 2 for recording a physiological electrical signal of a living being according to the invention comprises, in this instance, a radially internal first flange 26 and a radially external second flange 24, adjacent to the first flange 26 and extending at the level of a flanged portion 23 surrounding the body 25. The recording electrode 2 for recording a physiological electrical signal of a living being according to the invention further comprises a rigid mounting means 5, 51 fixed to the body 25, by bonding or vulcanizing for example. This mounting is performed at an edge of the electrical-contact rear surface, in this instance at the periphery of the electrical-contact rear surface, at the level of the first 26 and second 24 flanges. The rigid mounting means 5, 51 comprises a rigid cylindrical ring 5 fixed attached to the body 25 at the level of the second flange 24. A height of the ring 5 is such that, in this instance, this ring extends, on the rear, beyond the top of the rear surface 22 so as to project from said rear surface 22, while at the same time surrounding it from a distance. In this instance, the ring 5 has a rectangular cross section of which a width is less than the height. The ring 5 has a shape that complements the assembling means 41, here in the form of an annular groove, with which the ring 5 is intended to collaborate by being pushed onto same upon removable mounting of the recording electrode 2 for recording a physiological electrical signal of a living being according to the invention on the recording sensor 1 for recording a physiological electrical signal of a living being according to the invention. In addition, the rigid mounting means further comprises a stiffening washer 51 stiffening the recording electrode 2 for recording a physiological electrical signal of a living being according to the invention fixed attached to the body 25 at the level of the first flange 26. In one form of embodiment, the stiffening washer 51 is formed as one with the ring 5. The aforementioned removable mounting is performed by forcibly pushing the ring 5 into the annular groove of the rigid mounting means 41 until the rear surface 22 comes to press with electrical contact against the contact face 31 of the printed circuit 3. In one embodiment variant, the rear surface 222 is convex as illustrated in FIG. 2.

A second embodiment of the recording sensor 100 for recording a physiological electrical signal of a living being according to the invention will now be described with reference to this FIG. 2. Only the differences between the recording sensor 100 for recording a physiological electrical signal of a living being according to the invention and the sensor that has just been described with reference to FIG. 1 will be described.

The recording sensor 100 for recording a physiological electrical signal of a living being according to the invention comprises a casing 400 which comprises an assembling means 410 in the form of an annular groove surrounding the housing 42. The assembling means 410 here comprises a screw thread 411. To complement this, the recording electrode 200 for recording a physiological electrical signal of a living being according to the invention mounted removably on the recording sensor 100 for recording a physiological electrical signal of a living being according to the invention comprises the rigid mounting means which, in this instance, comprises a rigid ring 50 having a screw thread 52 that complements the screw thread 411. The ring 50 is cylindrical and fixed attached to the body 25 at the level of the second flange 24, by bonding or vulcanizing for example. Thus, the removable mounting of the recording electrode 200 for recording a physiological electrical signal of a living being according to the invention on the recording sensor 100 for recording a physiological electrical signal of a living being according to the invention is achieved by screwing and tightening until the rear surface 222 comes to press with electrical contact against the contact face 31 of the printed circuit 3. In this instance, the rear surface 222 is convex making it possible to ensure optimal electrical contact between said rear surface 222 and the contact face 31 of the printed circuit 3 when the recording electrode 200 for recording a physiological electrical signal of a living being according to the invention is tightened onto the recording sensor 100 for recording a physiological electrical signal of a living being according to the invention.

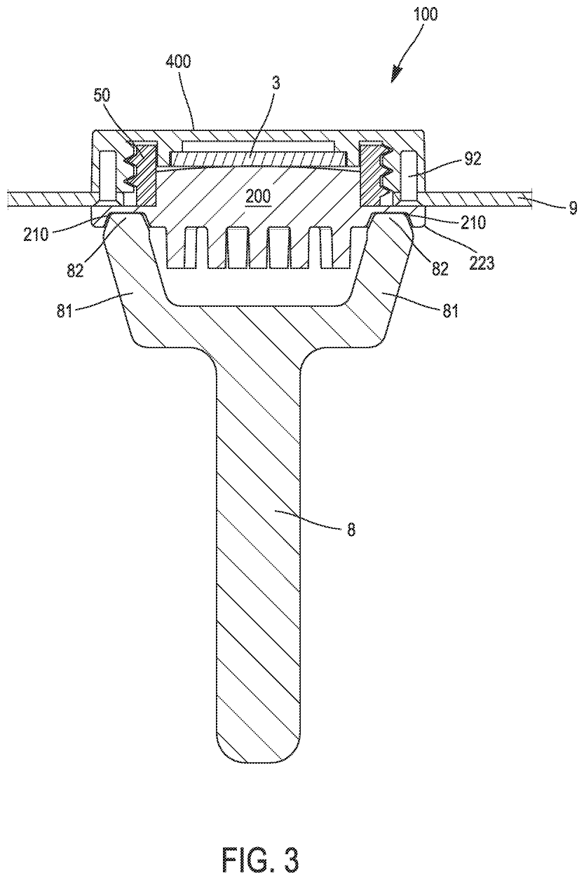

In addition, the body 25 comprises a flanged portion 223 defining the flange 24 and extending as a radial projection away from the center of the body 25 of the recording electrode 200 for recording a physiological electrical signal of a living being according to the invention. When the recording sensor 100 for recording a physiological electrical signal of a living being according to the invention is mounted on a device 9 for measuring a physiological electrical signal intended to be worn by a living being, it is sandwiched between the flanged portion 223 and the casing 400: the measurement device 9 for measuring a physiological electrical signal intended to be worn by a living being comprises a series of fixing holes 91 formed in line with a thickness of a lateral wall of the casing 400 and in which there are positioned fixing screws 92 (FIG. 3) engaging in the thickness of the lateral wall of the casing 400. The fitting of the recording electrode 200 for recording a physiological electrical signal of a living being according to the invention on the recording sensor 100 for recording a physiological electrical signal of a living being according to the invention is performed in such a way that the flanged portion 223 hides the fixing screws 92.

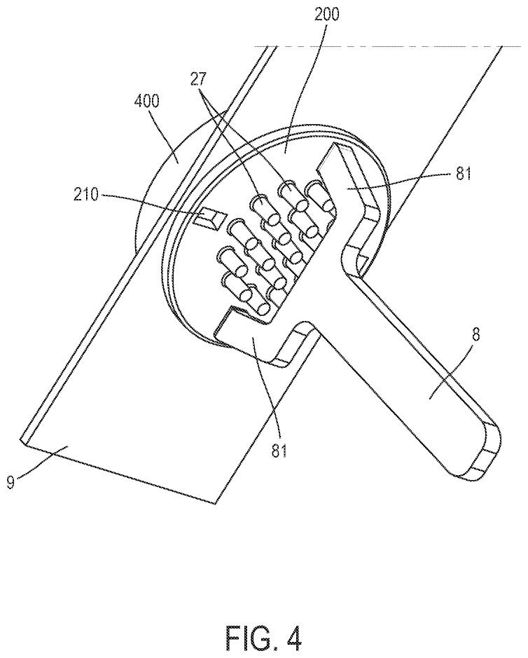

In order to make it possible easily to mount and demount the recording electrode 200 for recording a physiological electrical signal of a living being according to the invention, this electrode comprises, in this instance, a series of tool sockets 210. In this embodiment illustrated in FIGS. 3 and 4, the tool sockets 210 are recessed and made in a thickness of the flanged portion 223. In this instance, the tool sockets 210 have a substantially rectangular shape, oriented radially, and are four in number uniformly distributed on a circumference of the flanged portion 223. A tool 8 is provided for performing the removable mounting of the recording electrode 200 for recording a physiological electrical signal of a living being according to the invention. It comprises a series of branches 81 (in this instance two branches) each having an end 82 of a shape that complements the tool sockets 210. In a variant, the tool socket is a slot along a diameter of the recording electrode 2, 200 for recording a physiological electrical signal of a living being according to the invention, which slot is able to engage with a tool of the credit-card type for example. In another variant embodiment of the tool, the latter has a shape that complements the series of pins 27 so as to collaborate with same for mounting/demounting the recording electrode 2, 200 for recording a physiological electrical signal of a living being according to the invention.

In general, at least the body and the pins of the recording electrode 2, 200 for recording a physiological electrical signal of a living being according to the invention are made from a material which provides electrical conduction of an electrical signal from the free end of the pins (respectively from the front surface) as far as the rear surface of the body. In particular, this material may or may not be soft. For example, the material, in its soft version, is a polymer containing fillers that provide the electrical conduction. Other types of materials, soft or otherwise, allowing electrical conduction may be employed.

Of course, it is possible to make numerous modifications to the invention without in any way departing from the scope thereof.

1) An electrode for recording a physiological electrical signal of a living being made from an electrically conducting material and comprising a body comprising a front surface and a rear surface for electrical contact, wherein the electrode further comprises a rigid mounting means fixed to the body, at an edge of the electrical-contact rear surface and arranged in such a way as to achieve removable mounting of the electrode on a recording sensor.

2) The electrode as claimed in claim 1, wherein the material is a soft electrically conducting material of polymer type.

3) The electrode as claimed in claim 1, wherein the rear surface of the body is convex.

4) The electrode as claimed in claim 1, wherein the rigid mounting means comprises a cylindrical ring fixed attached to the body.

5) The electrode as claimed in claim 4, wherein the ring comprises a screw thread on a radially external surface.

6) The electrode as claimed in claim 4, wherein the ring projects from the rear surface of the body.

7) The electrode as claimed in claim 1, wherein the rigid mounting means comprises a stiffening washer.

8) The electrode as claimed in claim 1, wherein the electrode further comprises a tool socket allowing the removable mounting.

9) The electrode as claimed in claim 1, wherein it further comprises pins extending as a projection from the front surface.

10) A sensor for recording a physiological electrical signal of a living being comprising a casing comprising a housing and a printed circuit positioned in the housing, wherein it further comprises an electrode as claimed in claim 1 mounted removably on the casing, the rear surface pressing with electrical contact against a part of the printed circuit.

11) The sensor as claimed in claim 10, wherein the casing comprises an assembling means complementing the rigid mounting means of the electrode so that the electrode can be mounted removably on the sensor.

12) The sensor as claimed in claim 10, wherein the casing comprises a means forming shielding designed to achieve electromagnetic isolation.

13) A device for measuring a physiological electrical signal intended to be worn by a living being, wherein it comprises at least one sensor as claimed in claim 10.
